(19) United States
(12) Patent Application Publication
(10) Pub. No.: US 2024/0257936 A1
(43) Pub. Date: Aug. 1, 2024

(54) FEEDBACK SYSTEM AND METHOD FOR A DRUG PRODUCT

(71) Applicant: AMGEN INC., Thousand Oaks, CA (US)

(72) Inventors: Seemi Kazmi, Thousand Oaks, CA (US); Dan Picker, Thousand Oaks, CA (US); Gregory Thomas Nowak, Somerville, MA (US); Mitun Pragji, Thousand Oaks, CA (US)

(21) Appl. No.: 18/561,341

(22) PCT Filed: May 10, 2022
(86) PCT No.: PCT/US22/28430

§ 371 Date: Nov. 16, 2023

Related U.S. Application Data
(60) Provisional application No. 63/318,385, filed on Mar. 9, 2022.
(60) Provisional application No. 63/189,414, filed on May 17, 2021.

(51) Int. Cl. G16H 20/17; A61M 5/20; G16H 10/60; G16H 15/00; G16H 40/63; G16H 80/00

(52) U.S. Cl. CPC G16H 20/17; A61M 5/20; G16H 10/60; G16H 15/00; G16H 40/63; G16H 80/00; A61M 2205/3592; A61M 2205/581

(57) ABSTRACT

A reminder device includes a body having a touch screen and a button, a motion sensor, an audible indicator for generating an audible alarm, and a memory storing a dosing schedule and a reminder schedule. A processor is configured to execute logic stored on the memory to cause: (A) the touch screen to display a countdown timer for reminding the user of the at least one date for the dose, the countdown timer updating as the at least one date for the dose approaches and comprising a visible indicator, activated upon actuation of the button and sensing motion via the motion sensor, and (B) the audible alarm to activate at the at least one date for the dose. So configured, the motion sensor and alarms operate in a vicinity of storage of the drug product, enabling a user easily take the drug product upon using the reminder device.

### CROSS-REFERENCE TO RELATED APPLICATIONS

Priority is claimed to U.S. Provisional Patent Application No. 63/189,414, filed May 17, 2021, and U.S. Provisional Patent Application No. 63/318,385, filed Mar. 9, 2022, the entire contents of each of which are hereby incorporated by reference herein.

### FIELD OF THE DISCLOSURE

The present disclosure generally relates to reminder systems for drug products and, more particularly, to a customized system and method of managing reminders for a user to dose a drug product and providing feedback to the user as to the status of the dose.

### BACKGROUND

Non-daily medication doses, such as injections, are difficult to remember in the context of patients' busy lives and other health regimens. Patients have a range of lifestyles from more organized to unstructured routines. As such, it is common for many patients to inadvertently skip a dose, stretch a dose, and/or discontinue a dose of a needed medication. Often times, such patients skip or miss a dose of a needed medication at the last minute without an intention to skip. Typically, this problem is identified within the first few months of a given therapy, as patients are largely stretching their medication in this scenario. Patients with such unplanned skipping or stretching of doses are often younger, tend to be busier, and typically perceive a dosing routine as a burden, making it more likely for such patients to miss their needed doses. Moreover, existing reminder systems are not “life proof.” In other words, such existing mechanisms are easy to ignore in that the reminder merely ties a dose day to a consistent day and time of the week, and are often in the form of wall calendar and/or digital or computer phone reminder.

Such non-adherence in routinely taking needed medications is a large problem for patients, healthcare providers, and the pharmaceutical industry. Some studies have shown that up to 63% of patients are non-adherent for taking medications for chronic conditions in their first year of therapy. In addition, approximately 20% of hospital and long-term care facility admissions are related to non-adherence of medications. Further, studies have also shown that there is approximately $300 billion dollars in US annual excess costs due to medication non-adherence and approximately $188 billion dollars of biopharma revenue loss due to medication non-adherence.

Generally, the most common reason for non-adherence is the forgetfulness of patients. The problem is the greatest among patients who are most involved in their own care. Specifically, patients who take their medications at home are most likely to be non-adherent to a scheduled dose of needed medication.

In addition, many drug delivery devices, such as autoinjectors, must also provide feedback to the user as to the status of the dose once the patient begins taking the dose of needed medication, for example. Specifically, and in one example, the drug delivery device provides signals to the user during the dose for the user to gauge progress. These functions are most cost effectively done with visual, audible, and sometimes tactile mechanisms. Historically, when it was desired to improve the feedback function, low energy Bluetooth devices were relied on to allow a smart user device to connect with the drug delivery device, such as the autoinjector. However, these connections suffer from added complexity, costs, and often the need for chemical batteries, which may be hazardous to dispose of and represent a potential weak point from a reliability perspective.

### SUMMARY

In accordance with a first exemplary aspect, a reminder device for reminding a user when to dose a drug product is disclosed. The reminder device comprises a body having a touch screen and a button, a motion sensor carried by the body, an audible indicator carried by the body for generating an audible alarm, and a memory carried by the body and storing a dosing schedule and a reminder schedule. Each of the dosing schedule and the reminder schedule are based on user input via the touch screen, and the dosing schedule includes at least one date for a dose of the drug product, and the reminder schedule includes at least one time for activating the audible alarm. The reminder device further comprises a processor carried by the body and configured to access the dosing schedule and the reminder schedule and execute logic stored on the memory to cause the touch screen to display a countdown timer for reminder the user of the at least one date for the dose, the countdown timer updating as the at least one date for the dose approaches and comprising a visible indicator that is activated: (1) upon actuation of the button on the body; or (2) automatically at a set time before the at least one date of the dose and upon sensing motion via the motion sensor. In addition, the processor also executes logic stored on the memory to cause the audible alarm to activate at the at least one time for the dose.

In accordance with a second exemplary aspect, a reminder system for reminding a user when to dose a drug product is disclosed. The reminder system comprises a wireless network, at least one remote database operatively coupled to the wireless network, and a reminder device operatively coupled to the wireless network. The reminder device comprises a body having a touch screen and a button, a motion sensor carried by the body, an audible indicator carried by the body for generating an audible alarm, and a memory carried by the body and storing a dosing schedule and a reminder schedule. Each of the dosing schedule and the reminder schedule are based on user input via the touch screen, and the dosing schedule includes at least one date for a dose of the drug product, and the reminder schedule includes at least one time for activating the audible alarm. The reminder device further comprises a processor carried by the body and configured to access the dosing schedule and the reminder schedule and execute logic stored on the memory to cause: (A) the touch screen to display a countdown timer for reminder the user of the at least one date for the dose, the countdown timer updating as the at least one date for the dose approaches and comprising a visible indicator that is activated: (1) upon actuation of the button on the body; or (2) automatically at a set time before the at least one date of the dose and upon sensing motion via the motion sensor; and (B) the audible alarm to activate at the at least one time for the dose.

In accordance with a third exemplary aspect, a method of managing reminders for a user to dose a drug product comprises, upon actuating a reminder device, displaying a countdown timer on a touch screen of the reminder device, the countdown timer for reminding a user of at least one date for a dose of a drug product set by a user. The method also comprises updating the countdown timer as the at least one date for the dose approaches. The method also includes activating a visible indicator of the countdown timer: (1) upon actuating a button of the reminder device; and (2) automatically at a set time before the at least one date for the dose and upon sensing motion via motion sensor. The method still also includes activating an audible alarm of an audible indicator of the reminder device at a time set by the user.

In further accordance with any one of the foregoing exemplary aspects, the reminder device, the reminder system and/or method of managing reminders for a user to dose a drug product may further include any one or more of the following preferred forms.

In accordance with one preferred form, after the dose is due, the logic may be further executable by the processor to automatically change the touch screen to a color indicating the dose is past due.

In accordance with another preferred form, the body may include one or more of: (1) a magnet adapted to attach to a surface of a refrigerated compartment storing the drug product; or (2) a surface adapted to be coupled to a stand disposed near the drug product.

In accordance with another preferred form, each of the dosing schedule and the reminder schedule may include a frequency, such as weekly, every two weeks, monthly, twice weekly, once every number of months, or once every number of days, the number able to be set to a desired value

In accordance with another preferred form, the reminder device may further comprise a user-configured wireless communication module for wireless transmission of a text message reminder to a user device, and the text message may be transmitted one or more of: (1) at the date the dose is due, or (2) when the dose is past due.

In accordance with yet another preferred form, the reminder device may further comprise a log of information about depersonalized user actions collected by the processor and stored in the memory, and a user-configured wireless communication module for wireless transfer of the log of information to a remote database.

In accordance with yet another preferred form, the reminder device may further comprise a temperature timer configured to alert the user when the drug product has been out of a refrigerated compartment for a sufficient time, allowing the drug product to reach a desired temperature, such as room temperature.

In accordance with another preferred form, wherein, after the dose of the drug product is indicated to be taken, the logic may be further executable by the processor to provide an option to reorder on the touch screen, and the option to reorder may be activated based on user input via the touch screen.

In accordance with another preferred form, the at least one remote database of the reminder system may comprise a database for receiving a log of information including depersonalized actions collected by the processor of the reminder device or a database for receiving personalized used actions collected by the processor. Each of the databases may prepare reports for the logs of information.

In accordance with another preferred form, the reminder system may further comprise a user device. The user device may be coupled to the wireless network and may be for receiving a text message reminder from the reminder device. The text message reminder may be sent one or more of at the at least one date for the dose or when the dose is past due.

In accordance with yet another form, the method may further comprise storing, via a memory of the reminder device, one or more of a dosing schedule and a reminder schedule based on input from the user. The dosing schedule may include the at least one date for a dose of the drug product, and the reminder schedule may include the time for activating the audible alarm.

In accordance with yet another form, the method may further comprise automatically changing the touch screen of the reminder device to a color indicating the dose is past due, providing another reminder to the user.

In accordance with yet another form, the method may further comprise transmitting, via a processor of the reminder device, a text message reminder to a user device via a wireless network at the at least one date for the dose or when the dose is past due, and the text message reminder may include the at least one date for the dose.

In accordance with another form, the method may comprise actuating the touch screen on the reminder device in response to the audible alarm to initiate a snooze function for the audible alarm and for a period of time, and automatically reactivating the audible alarm after the period of time expires.

In accordance with yet another form, the method may further comprise activating a temperature timer before the at least one date for the dose, the temperature timer configured to alert the user when the drug product has been out of a refrigerated compartment for a sufficient time, allowing the drug product to reach a desired temperature, such as room temperature.

In accordance with another exemplary aspect, a feedback system for use with a dose of a drug product comprises a wireless network, a drug delivery device including a body and an audible indicator carried by the body and for generating one or more audible signals, and an intermediary device operatively coupled to the drug delivery device and the wireless network. The intermediary device includes a body having a microphone configured to amplify the one or more audible signals generated by the drug delivery device; at least one feedback mechanism carried by the body, a memory and a processor carried by the body. The processor is configured to execute logic stored on the memory to cause the at least one feedback mechanism to provide feedback to the user about a dose of the drug product based on the one or more audible signals generated by the drug delivery device, and the feedback includes one or more of an audio indication or a visual indication.

In accordance with another exemplary aspect, a method of providing feedback to a user about a dose of a drug product comprises receiving, via an intermediary device, one or more audible signals generated by a drug delivery device, and the intermediary device is operatively coupled to a wireless network and the drug delivery device. In addition, the method further comprises providing feedback to a user about a dose of a drug product of the drug delivery device via at least one feedback mechanism of the intermediary device based on the one or more audible signals. The feedback includes one or more of an audio indication or a visual indication.

### BRIEF DESCRIPTION OF THE DRAWINGS

The above needs are at least partially met through provision of the embodiments described in the following detailed description, particularly when studied in conjunction with the drawings, wherein:

FIG. 1 is a perspective view of a reminder system of the present disclosure;

FIG. 2 is block diagram of portions of the reminder system of FIG. 1;

FIG. 3 is a perspective view of a reminder device of the reminder system according to one embodiment;

FIG. 4A is a perspective view of a touch screen of the reminder device of the present disclosure;

FIG. 4B is a view of optional displays on a touch screen of the reminder device of the present disclosure, the optional displays indicating a stage of the reminder device of the present disclosure;

FIG. 5 is a flow chart illustrating a method of setting up a wireless communication module with the reminder device of the present disclosure;

FIG. 6 is a flow chart illustrating a method of customizing, including setting an actual time and date, the reminder device of the present disclosure;

FIG. 7 is a flow chart illustrating a method of setting a dosing schedule for a dose of a drug product and a reminder schedule with the reminder device of the present disclosure;

FIG. 8 is a flow chart illustrating a method of selecting features of a reminder of the reminder system of the present disclosure;

FIG. 9 is a flow chart illustrating a method of selecting a text reminder with the reminder device of the present disclosure;

FIG. 10 is flow chart illustrating a countdown displayed on a touch screen of the reminder device during different periods of time before the selected date and time of a dose of the drug product;

FIG. 11 is a block diagram of a feedback system of the present disclosure;

FIG. 12 is another block diagram of the feedback system of FIG. 11, with the reminder device;

FIG. 13 is a block diagram of portions of the feedback system of FIG. 12;

FIG. 14 is an exemplary visual indication of an intermediary device of the feedback system of FIGS. 11 and 12;

FIG. 15 is another exemplary visual indication of the intermediary device of the feedback system of FIGS. 11 and 12;

FIG. 16 is another exemplary visual indication of the intermediary device of the feedback system of FIGS. 11 and 12;

FIG. 17 is another exemplary visual indication of the intermediary device of the feedback system of FIGS. 11 and 12; and

FIG. 18 is a listing of other exemplary visual indications of the feedback system of FIGS. 11 and 12 of the present disclosure.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Generally, a system and method are disclosed that utilize a reminder device for reminding a user when to dose a drug product. The reminder device is designed to aid patients in managing a non-daily, self-injectable drug product, such as a biologic, and provides multiple reminders, including visual and audible reminders, and an optional text message reminder. The reminder device may be attached to a refrigerated compartment storing the drug product or disposed near the drug product to facilitate ease of injection after the reminders. More specifically, the reminder device includes a body having a touch screen, a button, a motion sensor carried by the body, an audible alarm, a power source, such as a battery, and a memory for storing the dosing and reminder schedules, and a processor to execute logic stored on the memory to cause the touch screen to display a countdown timer for reminding the user of at least one date for the dose of the drug product. The countdown timer is updated as the at least one date for the dose approaches and comprises a visible indicator, which is activated upon actuation of a button or automatically at a set time before the at least one time for the dose and upon sensing motion via the motion sensor. In addition, the processor also executes logic stored on the memory to cause the audible alarm to activate at the at least one time for the dose. While the user is required to input the dosing and reminder schedules, any specific drug product is never entered into and/or associated with the reminder device or system of the present disclosure. Moreover, the dosing and reminder schedules set by the user are for a single drug product, and the reminder device and system do not accommodate dosing and reminder schedules for multiple drug products.

In addition, a system and method are disclosed that utilize an existing, wireless intermediary device, such as the reminder device or another smart device, to provide feedback to the user about the dose of the drug product based on audible signals generated by a drug delivery device. More specifically, the system and method use existing audible feedback mechanisms on drug delivery devices as a connection to the intermediary device, e.g., the reminder device. The intermediary device is able to discern at a minimum audible signals from the drug delivery device associated with a start and an end of the dose of the drug product, such as an injection, and provide feedback to the user based on the audible signals from the drug delivery device. As a result, the intermediary device augments feedback in form of the audible signals from the drug delivery device with louder and/or more descriptive feedback to the user.

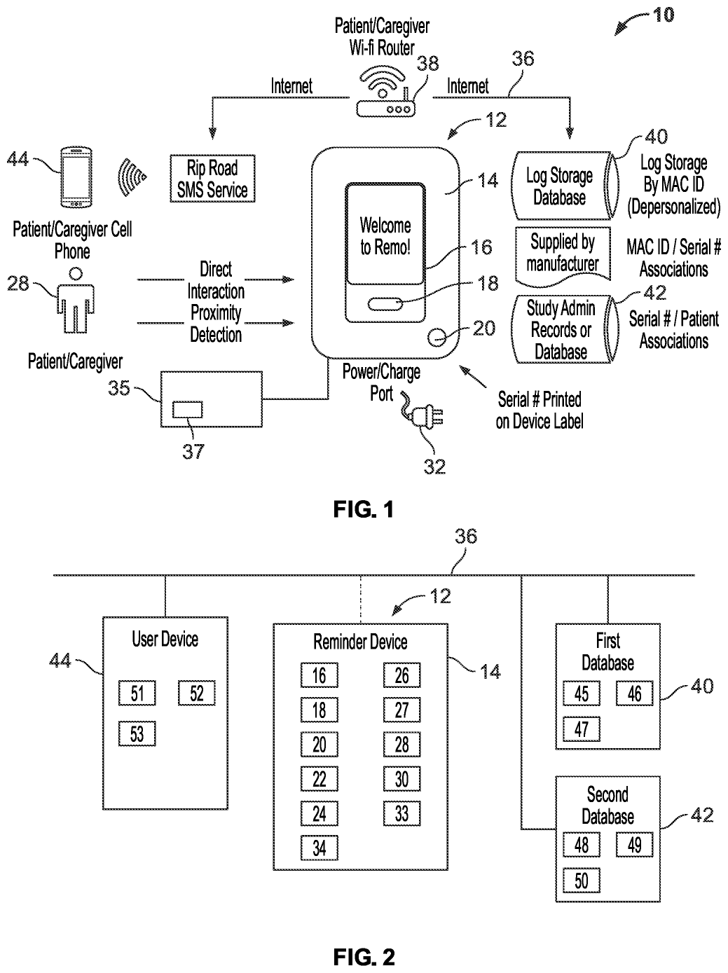

Referring now to FIG. 1, a reminder system 10 according to the present disclosure is depicted. The reminder system 10 includes the reminder device 12 having a body 14 with a touch screen 16 and a button 18. In this example, the touch screen 16 is a color touch screen. The reminder device 12 also includes a motion sensor 20 carried by the body 14. While the motion sensor 20 is disposed on a lower area of the body 14, it will be appreciated that the motion sensor 20 may be disposed on any part of the reminder device 12 and still fall within the scope of the present disclosure.

As depicted in FIG. 2, the reminder device 12 further includes an audible indicator 22 carried by the body 14 and for generating an audible alarm, a processor 24, and a memory 26, and each of the processor 24 and memory are also carried by the body 14. In addition, the memory 26 stores a dosing schedule and a reminder schedule, and each of the dosing schedule and the reminder schedule are based on user input provided via the touch screen 16 of the reminder device 12, as also described more below. Further, the dosing schedule includes at least one date for the dose inputted by the user, and the reminder schedule includes at least one time for activating the audible alarm of the audible indicator 22. The processor 24 is configured to access the dosing schedule and the reminder schedule and execute logic stored on the memory 26 to cause: (A) the touch screen 16 to display a countdown timer 27 for reminding the user of the at least one date for the dose. As explained more below, the countdown timer 27 updates as the at least one date for the dose approaches and comprises a visible indicator 28. The visible indicator 28 may include text or a color on a display of the touch screen 16, as explained more below. In addition, the visible indicator 28 may be activated upon actuation of the button 18 on the body 14 of the reminder device 12. Further, the visible indicator 28 may also be automatically activated at a set time before the at least one date of the dose and upon sensing motion via the motion sensor 20. Still further, the processor 24 may also execute logic stored on the memory 26 to cause the audible alarm of the audible indicator 22 to activate at the least one time for the dose, as also explained more below.

In addition, the reminder device 12 includes a power source 30, which may include a battery or a wired connection, a power/charge port 32, a temperature timer 33, and a magnet or magnetic portion 34. The power/charging port 32 is used for powering the power source 30, such as a battery, of the reminder device 12, and no data can be accessed from the reminder device 12 via this port 32. Further, the magnet or magnetic portion 34 of the reminder device 12 may be attached to a surface of a refrigerated compartment 35 storing the drug product 37, such as the drug product for injection. The temperature timer 33 may be activated for a period of time, such as 30 minutes, when the audible alarm 22 is activated, enabling the drug product 37 to be prepared for the dose, as also explained more below. In addition, the temperature timer 33 is configured to alert the user when the drug product has been out of the refrigerated compartment 35 for a sufficient time, allowing the drug product to reach a desired temperature, such as room temperature.

Referring back to FIG. 1, the reminder system 10 also includes a wireless network 36, and the reminder device 12 may be operatively coupled to the wireless network 36 via a Wi-Fi router 38 of the user. The reminder system 10 also includes at least one remote database operatively coupled to the wireless network 36. Specifically, the reminder device 12 supports an optional Wi-Fi connection that allows it to connect to an SMS server and the at least one remote database. In this example, the reminder system 10 includes a first database 40, such as a cloud mine database, for receiving a log of information about depersonalized user actions collected by the processor 24 and stored in the memory 26 of the reminder device 12. In addition, the reminder system 10 may also include a second database 42 for receiving a log of information about personalized user actions again collected by the processor 24 and stored in the memory 26 of the reminder device 12.

In one example, the logs of information include usage logs that allow administrators to understand patients' usage of the reminder device 12, including how compliant they have been in taking their drug products. The data included in the logs of information may include, but is not limited to, several events, such as a dose ready alarm, a missed alarm, a timer is ready, a timer missed alarm, a text sent regarding drug product dose, a low battery indicator, and if Wi-Fi setup failed. The data in the logs of information may also include data about user actions, such as when did the first start up/initialization occur, when a timer was used, if an alarm was dismissed, if a user actuated a “take now” button on the touch screen 16, if the reminder device 12 was plugged into a charger, and if a Wi-Fi connection was established. Such events and actions are explained more below.

As depicted in FIG. 2, the first database 40 includes a processor 45, a memory 46, and a network interface 47, and the second database 42 also includes a processor 48, a memory 49, and a network interface 50. So configured, the processors 45, 48 of each of the first and second databases 40, 42 receive the logs of information from the reminder device 12 and may generate reports based on the collected information. As also depicted in FIG. 2, and as will be understood, the user device 44 of the reminder system 10 also includes at least a processor 51, a memory 52, and a network interface 53 operatively coupled to the wireless network 36 and through which the text reminder from the reminder device 12 is transmitted.

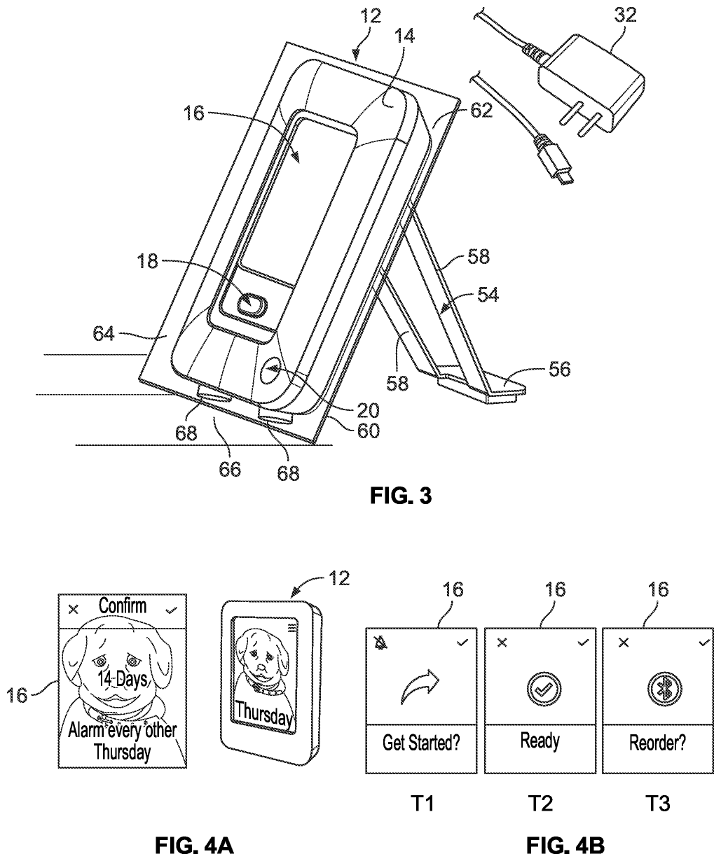

Referring now to FIG. 3, the reminder device 12 of the reminder system 10 is depicted. In this example, the reminder device 12 is not coupled to the refrigerated compartment 35 via the magnet 34 of the reminder device 12, as in FIG. 1. Instead, the reminder device 12 is disposed in a stand 54. The stand 54 includes a base 56, a pair of support members 58 upwardly extending from and connected to the base 56, and a body 60 having a first end 62 to which the pair of support members 58 are connected and second end 64 adapted to contact a surface 66 disposed near the storage of the drug product 37. So configured, the body 60 extends downwardly from the pair of support members 58. The second end 62 of the body 60 of the base 56 also includes a pair of projections 68. The reminder device 12 is disposed on the body 60, such that one end of the reminder device 12 is maintained in place on the body 60 via the pair of projections 68, for example. The stand 54 may take the form of various other shapes, sizes, and examples that function in a manner similar to or the same as the stand 54 and still fall within the scope of the present disclosure. The stand 54 helps dispose the touch screen 16 and the reminder device 12 at an angle, instead of a horizontal position on a relatively flat surface, so that the touch screen 16 is readily visible to the user.

Referring now to FIGS. 4A and 4B, exemplary displays on the touch screen 16 of the reminder device 12 of the present disclosure are depicted. Specifically, in FIG. 4A, a perspective view of the touch screen 16 of the reminder device 12 is depicted after a user has inputted a date and time for the dose of the drug product and a reminder for activation of the audible alarm of the audible indicator 22. As illustrated, in this example, the user has inputted that the dose of the drug product should be taken every 14 days and the audible alarm of the audible indicator 22 is activated every other Thursday. The user also selected a custom screen saver (e.g., of her pet) as displayed in the background of the touch screen 16 of the reminder device 12. As will be appreciated, the displayed screen saver is an example only, and various other alternative screen savers may alternatively be used and customized on the touch screen 16 of the reminder device 12. FIG. 4B depicts displays on the touch screen 16 of the reminder device 12, each of which is displayed at a different time during use of the reminder device 12. For example, at a time T1, the touch screen 16 displays text asking a user if he or she wishes to “get started”. If yes, the user contacts the touch screen 16 at the location of the arrow displayed thereon and is prompted to input various settings, a dosing schedule, and a reminder schedule, as explained more below. Once all of the required information is inputted into the reminder device via the touch screen 16 by the user, the touch screen 16 displays text indicating the reminder device 12 is ready for operation at a time T2. The reminder device 12 then provides reminders based on both the user inputted dosing schedule and the reminder schedule until the dose of the drug product 37 has been taken, as inputted by the user. Once this action is complete, and at a time T3, the touch screen 16 of the reminder device 12 then displays text asking if a user desires to reorder the drug product 37. The reordering may be conducted via the reminder device 12 and/or via the user device 44 via the wireless network 36, for example. Alternatively and/or additionally, the drug product may be reordered using any other known means for reordering the drug product.

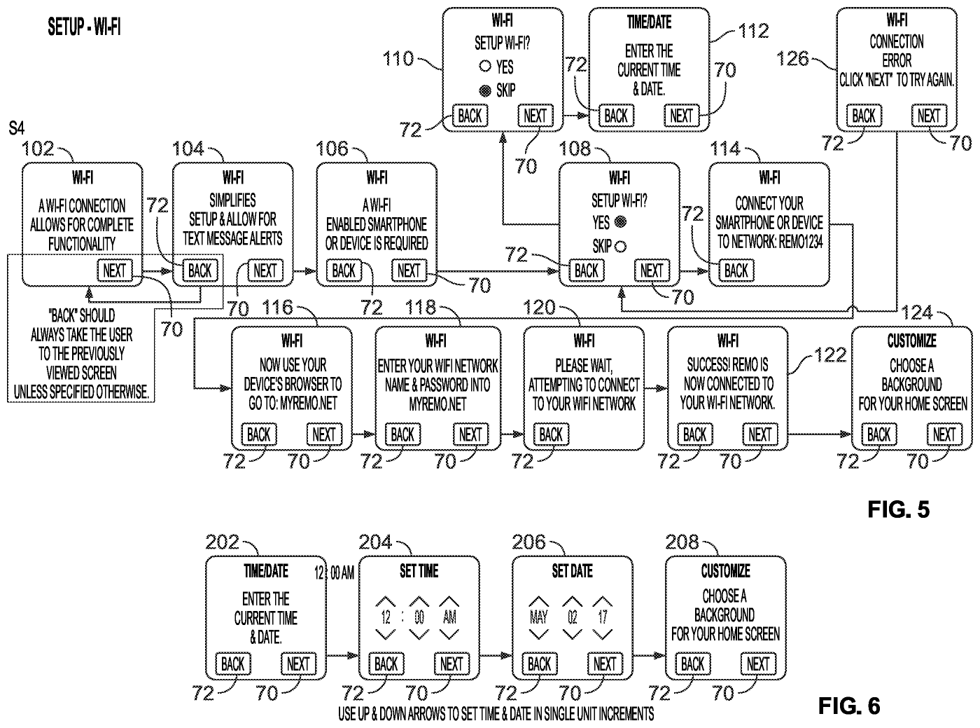

Referring now to FIGS. 5-9, various flow charts depicting processes for setting up and customizing the reminder device 12 of the present disclosure are depicted. Referring first to FIG. 5A, a process of setting up and establishing a wireless communication module with the reminder device 12 of the present disclosure is depicted. Specifically, and in this example, upon powering on the reminder device 12, the touch screen 16 first indicates that a Wi-Fi connection allows for complete functionality in step 102. If the user touches the “next” touch button 70 displayed on the touch screen 16, the touch screen 16 then indicates in step 104 that Wi-Fi simplifies set up and allows for text messaging alerts. The user may again via the touch screen 16 contact the “next” touch button to advance the display on the touch screen 16 in step 106. In step 106, the touch screen 16 indicates a Wi-Fi enabled smart phone or device is required. Upon contacting the “next” touch button 70 again on this display, the touch screen 16 then asks the user if a Wi-Fi connection is desired in step 108. If the user selects the SKIP button as in step 110, the touch screen 16 then displays a prompt for the user to enter the current time and date in step 112, which is explained more below in FIG. 6. However, if the user selected the YES button as in step 108, the touch screen 16 displays the text in step 114 to connect the user's device to a specific network. Upon doing so, the touch screen 16 then displays text indicating the user should utilize the browser of the user device 44 to go to a website of the reminder device 12, such as MyRemo.net. Next, in step 118 the touch screen 16 prompts the user to enter his or her Wi-Fi network name and password into the website of the reminder device 12, thereby establishing a user-configured wireless communication module with the reminder device 12, for example. As indicated in step 120, the touch screen 16 indicates to the user that the reminder device 12 is attempting to connect to the wireless network 36, for example, and then indicates on the touch screen 16 when the reminder device 12 is connected, as in step 122. If there is an error in trying to connect at any point in time, the touch screen 16 will display an error message, as in step 126, which then routes the user back to the display on the touch screen 16 of the reminder device 12 in step 108. Once the wireless communication module is established with the reminder device 16, the user may further customize the touch screen 16 of the reminder device 12, such as by selecting a background or screen saver, as indicated in step 124. It will be appreciated that the user may at any time contact the “back” touch button 72 on any of the displays of the touch screen 16 in the steps of FIG. 5, which will take the user to the previously viewed display on the touch screen 16 of the reminder device 12.

Referring now to FIG. 6, a flow chart illustrating an exemplary process for setting up the current time and date on the reminder device 12 is depicted. Specifically, in step 202, the touch screen 16 first displays text prompting the user to enter the current time and date. Upon selecting the “next” touch button 70 on the touch screen 16 of the reminder device 12, the touch screen 16 then provides a prompt for the user to enter the current time in step 204. Specifically, the user utilizes the up and down arrows 76 to set the current time. Once completed, the user again selects the “next” touch button 70 to advance to the display on the touch screen 16 in step 206. In this step, the user is then prompted to enter the current date, again using the up and down arrows 76 to do so. Once complete, and upon contacting the “next” touch button 70, the touch screen 16 provides another opportunity for the user to customize the reminder device 12, such as by selecting a background for the touch screen 16 of the reminder device 12 in step 208.

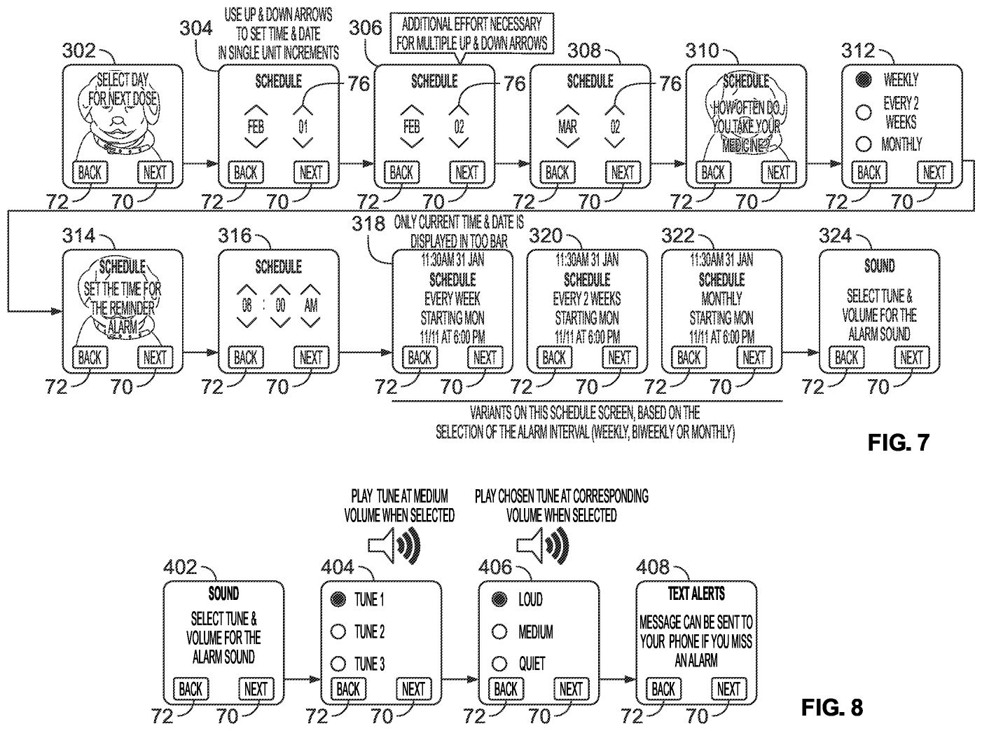

As depicted in FIG. 7, and after the settings on the reminder device 12 have been set by the user, the touch screen 16 displays prompts for setting a dosing schedule for a dose of the drug product and a reminder schedule for the reminder device 12. Specifically, the touch screen 16 first prompts the user to select a date for the dose in step 302. Then the touch screen 16 prompts the user to select a day for the dosing schedule using the up and down arrows 76 provided on the display of the touch screen 16, as in steps 304, 306, and 308. In particular, the touch screen 16 includes a month and a day within the month, e.g., the date, both of which are individually selected via the up and down arrows 76. For example, in step 306 the user moved the up and down arrows 76 in step 304 to change the day in the month of February from 01 to 02 in step 306. In step 308, the user moved the up and down arrows 76 to change the month from February to March. In step 310, the touch screen 16 next provides a prompt regarding how often the user takes his or her drug product. Upon actuation of the “next” button by the user in step 310, the touch screen 16 provides three options regarding the frequency of the dose of the drug product. Specifically, in step 312, the touch screen 16 provides a button indicating the drug product is taken weekly, another button indicating the drug product is taken every two weeks, i.e., bi-weekly, and a third button indicating the drug product is taken monthly. The user next selects one of the buttons corresponding to the accurate frequency of the dose of the drug to finalize the dosing schedule for the reminder device 12. In this example, the user selected the button corresponding to the weekly frequency, but it will be understood that a user may alternatively select either of the other buttons to set the dosing schedule. While not depicted, it will also be understood that a user may input a frequency of dose the same as or different from the three example frequencies listed in step 312 via a numerical input, such as by way of his or her user device 44 operably coupled to the reminder device 12 and still fall within the scope of the present disclosure. More specifically, and in one example, the frequency may be weekly, bi-weekly, monthly, twice weekly, once every number of months, or once every number of days, and the user is able to set the number to a desired value, such as 2, 3 or 4, for example. It will be understood that any of these example frequencies may be used and still fall within the scope of the present disclosure.

After the dosing schedule for the dose of the drug product has been set, the touch screen 16 then displays prompts for setting a reminder schedule for the reminder device 12, as also depicted in FIG. 7. Specifically, in step 314, the touch screen 16 first displays a prompt for the user to set the time for a reminder for an alarm, such as the audible alarm of the audible indictor 22 of the reminder device 12. Upon actuating the “next” button on the touch screen 16 to set the time for the reminder, the touch screen 16 next prompts the user to select a time using the up and down arrows 76, as depicted in step 316. In steps 318, 320, and 322, the touch screen 16 displays a prompt including a different frequency for the reminder for the alarm. Specifically, in step 318 the touch screen 16 displays a prompt indicating the reminder will occur every week starting at a specific date and time. In step 320, the touch screen 16 displays a prompt indicating the reminder will occur every two weeks, i.e., bi-weekly, starting at a specific date and time. In step 322, the touch screen 16 displays a prompt indicating the reminder will occur each month starting at a specific date and time. As indicated in each of the touch screens for steps 318, 320, and 322, the current time and date are displayed in a top area of the touch screen 16. Upon selection of one of the three times provided in steps 318, 320 or 322 for the reminder to occur, the touch screen 16 next displays a prompt for selecting a sound for the audible alarm of the audible indicator 22 in step 324, as explained in FIG. 8. In each of the steps of selecting the reminder schedule in FIG. 7, the touch screen 16 displays the “back” and the “next” buttons 70, 72, which the user may contact to move between displays of the touch screen 16, as needed when selecting and setting the reminder schedule for the reminder device 12.

Referring now to FIG. 8, a process for selecting a sound for the audible alarm of the audible indicator 22 of the reminder device 12 is depicted. Specifically, in step 402, the touch screen 16 initially displays a prompt indicating the user may select a tune or song and a volume for the audible alarm. In step 404, the touch screen 16 displays a prompt including three “tunes” for selection, one of which may be selected by the user. While three “tunes” for selection are displayed in step 404, it will be appreciated that a greater amount of songs may additionally be accessed and selected and still fall within the scope of the present disclosure. After the song or tune for the audible alarm is selected, the touch screen 16 next displays a prompt listing three volume levels for the song when the audible alarm of the audible indicator 22 is activated. Specifically, the three volume levels listed include loud, medium, and quiet, and the user selects one of the three volumes displayed. Lastly, in step 408, the touch screen 16 next displays a prompt indicating that text messages may optionally be sent to the user's device 44, e.g., the smart phone, such as when the audible alarm of the audible indicator 22 is missed.

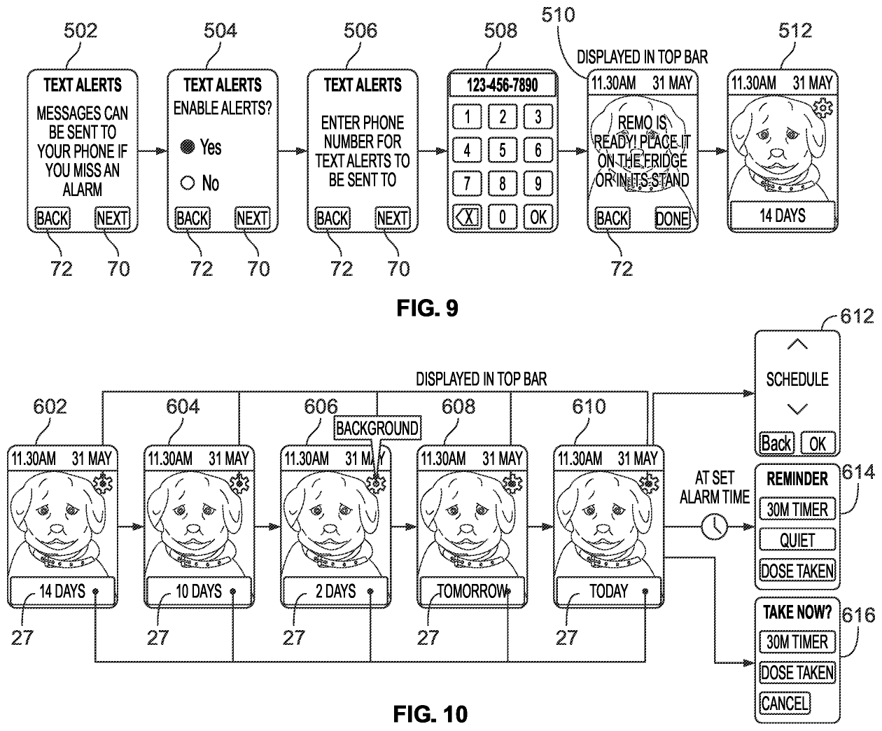

Referring now to FIG. 9, when the user selects the optional text message reminder, the touch screen 16 of the reminder device 12 includes additional prompts for setting the text message reminder. Specifically, upon selecting the “next” button 70 in step 502 of FIG. 9, the touch screen 16 displays a prompt to enable the text message reminder in step 504. Upon actuation of the “yes” button in step 504 to enable the text message reminder, the touch screen 16 next displays a prompt requiring the user to enter a phone number for the text message reminder to be sent in step 506. In step 508, the touch screen 16 displays a number keyboard for the user to enter the phone number. Once the phone number is entered, the touch screen 16 of the reminder device 12 indicates the reminder device 12 is ready for use and may be secured to the refrigerated compartment 35 (FIG. 1) or the stand 54 (FIG. 3), for example, in step 510. In this state, in step 512 the touch screen 16 of the reminder device 12 displays the selected background or screen saver, the actual date and time, and a countdown timer indicating a number of days until the dose of the drug product is due (based on the dosing schedule inputted).

More specifically, upon actuation of the button 18 of the reminder device 12, the touch screen 16 displays the countdown timer 27 for reminding the user of the set date for the dose of the drug product, as depicted in FIG. 10. Thus, a countdown timer 27 on the touch screen 16 is a visual reminder to the user. As shown in FIG. 10, the countdown timer 27 is updated as at least one date and time for dose approach. In addition, the countdown timer 27 includes the visible indicator 28 that is activated upon actuation of the button 18 (FIG. 1) of the reminder device 10. Further, the visible indicator 28 may also be automatically activated at a set time before the at least one date and time of the dose and upon sensing motion via the motion sensor 20. Said another way, the motion sensor 20 is activated once the countdown timer 27 reaches a time shortly before the at least one date of the dose, such as the day before or another time shortly before the dose due date. In the example of FIG. 10, the countdown timer 27 begins 14 days before the set date of the dose of the drug product in step 602. While not depicted in FIG. 10, the countdown timer 27 of the reminder device 12 typically counts in single day units. In step 604, the touch screen 16 of the reminder device 12 displays the countdown timer 27, indicating there are 10 days until the date of the dose and an associated reminder, such as the audible alarm of the audible indicator 22, for example. In step 606, the touch screen 16 of the reminder device 12 displays the updated countdown timer 27, indicating there are 2 days left. When the time reaches the day before the dose is due, the countdown timer 27 switches to “TOMORROW” as in step 608, and when the time reaches the actual day the dose is due, the countdown timer 27 switches to “TODAY” as depicted in step 610 of FIG. 10. In this example, the motion sensor 20 is activated when the countdown timer 27 reaches “TOMORROW” in step 608 or the day before the dose is due and remains activated when the countdown timer 27 reaches “TODAY” in step 610. In one example, if motion is detected by the motion sensor 20, the touch screen 16 of the reminder device 12 will turn on, e.g., be visible and/or activated, for 30 seconds and will not turn on again for another 10 minutes. Of course, it will be understood that such time frames may vary and be alternatively set to different values and still fall within the scope of the present disclosure.

As noted above, the visible indicator 28 of the countdown timer 27, such as texting indicating the time and/or days remaining in the countdown, may be automatically activated at a set time before the date for the dose and upon sensing motion via the motion sensor 20. In this example, the motion sensor 20 senses the motion of any user, such as the patient or a caregiver, with the range of the reminder device 12. However, it will be appreciated that a camera, such as a camera sensor, and facial recognition algorithms may alternatively be used with the reminder device 12 and/or the reminder system 10 to replace the motion sensor 20. The camera sensor may be trained to recognize only the patient taking the drug product. As a result, the visual indicator 28 of the countdown timer 27 would only activated after only motion of the actual patient is sensed, for example, limiting alarm and other proximity-based activity to the actual patient. This may be achieved using known artificial intelligence techniques, for example.

When the countdown timer 27 reaches “TODAY” as in step 610, the audible alarm of the audible indicator 22 of the reminder device 12 is activated according the audible alarm settings inputted by the user, as described above. In one example, the audible alarm continues for 15 seconds and the touch screen 16 simultaneously turns on, e.g., is visible, for 30 seconds. In addition, the touch screen 16 displays a schedule prompt to the user providing an opportunity to maintain the same dosing and reminder schedules initially input by the user or change the dosing and reminder schedules in step 612. If the user actuates the “back” button on the touch screen 16, the touch screen 16 displays the same options as provided in FIG. 7 and described above.

Further, the touch screen 16 also displays a reminder prompt, which provides three options for the user as depicted in step 614. Specifically, the reminder prompt on the touch screen 16 provides an option for activating the temperature timer 33 for a period of time, such as 30 minutes, after the audible alarm is activated, enabling the drug product to be prepared for the dose. It will be understood that the period of time may be greater or less than the 30 minutes and still fall within the scope of the present disclosure. In one example, preparing the drug product for injection includes removing the drug product 37 from the refrigerated compartment 35 and allowing the drug product 37 to be warmed to room temperature within the 30 minute time frame of the temperature timer 33. Further, once the prompt for the temperature timer 33 is selected, the touch screen 16 displays the timer in a countdown form, e.g., starting at 30 minutes, and provides an option for the user to dismiss the temperature timer at any time, e.g., such as if the drug product is prepared and ready for the dose, or indicate that the dose has been taken, which will stop the temperature timer 33 as well. If neither are selected, the temperature timer 33 will display the countdown timer 27 until the time expires and then sound an alarm, indicating the time of the temperature timer 33 has expired.

In step 614, the reminder prompt also lists the option of selecting “quiet” as a snooze feature of the reminder device 12 upon activation of the audible alarm 22. As will be understood, if the user selects “quiet” on the touch screen 16, the audible alarm is silenced for a period of time and the reactivated to alert the user again that it is time for the dose of the drug product. As a final option in step 614, the reminder prompt on the touch screen 16 lists the option of selecting “dose taken.” If the user selects “dose taken,” the motion sensor 20 is deactivated and the countdown timer 27 is reset on the display of the touch screen 16 according to the dosing schedule and the reminder schedule set by the user.

Still further, the touch screen 16 of the reminder device 12 also depicts a “take now” prompt as in step 616. Specifically, the “take now” prompt of the touch screen 16 again provides the option for activating the temperature timer 33, as described above relative to step 614. In addition, the “take now” prompt lists the option of selecting “dose taken,” as also described above relative to step 614.

In view of the foregoing, it will be understood that the reminder device 12 and reminder system 10 may operate according to the following method. Specifically, a method of managing reminders for a drug product may include, upon actuating the reminder device 12, displaying the countdown timer 17 on the touch screen 16 of the reminder device 12, the countdown timer 27 for reminding the user of at least one date for a dose of a drug product set by the user. The method may also include updating the countdown timer 27 as the at least one date of the dose approaches. The method may further include activating the visible indicator 28 of the countdown timer 27: (1) upon actuating the button 18 of the reminder device 12; or (2) automatically at a set time before the at least one date for the dose and upon sensing motion via the motion sensor 20. Still further, the method includes activating the audible alarm of the audible indicator 22 of the reminder device 22 at a time set by the user. In some examples, the method may also include storing, via the memory 26 of the reminder device 12, one or more of the dosing schedule and the reminder schedule based on input from the user, and the dosing schedule includes the at least one date for a dose of the drug product, and the reminder schedule includes the time for activating the audible alarm. In addition, the method may further include automatically changing the touch screen 16 of the reminder device 12 to a color indicating the dose is past due, providing another reminder to the user. Further, the method may include transmitting, again via the processor 24 of the reminder device 12, a text message reminder to the user device 44 via the wireless network 36 at the at least one date for the dose or when the dose is past due. Still further, the method may include actuating the touch screen 16 on the reminder device 12 in response to the audible alarm 22 to initiate a snooze function for the audible alarm 22 and for a period of time, and automatically reactivating the audible alarm 22 after the period of time expires. Lastly, the method may further include activating the temperature timer 33 before the date and the time for the dose, enabling the drug product to be prepared for the dose. More specifically, and in one example, the temperature timer 33 is configured to alert the user when the drug product has been out of the refrigerated compartment 35 for a sufficient time, allowing the drug product to reach a desired temperature, such as room temperature.

At least in view of the foregoing, it will be appreciated that the reminder device 12, reminder system 10, and method described above have several advantages. For example, the reminder device 12 and reminder system 10 help patients take their drug products on time. Specifically, in at least one study, “on-time” dosing of a needed drug product improved 12% at three months and 18% at six months, and there was a simultaneous reduction in late and skipped doses using the reminder device 12. In addition, at least one study also found that 40% of patients took their drug product within the first hour after the audible alarm of the reminder device 12. Regarding adherence improvement, participants in the study reported between 58-69% medication adherence for their most recent four doses, such as injections, using the reminder device 12. In addition, by six months, patients reported between 70-86% adherence using the reminder device 12. Further, at least the audible alarm, the text message reminders, and the countdown timer 27 having the visible indicator 28 on the touch screen 16 helped patients take their drug products on time more frequently. As a result, patients are having greater success with dosing schedules and the reminder device 12 and methods are reducing the often perceived burden of the routine of taking non-daily medications at a certain time.

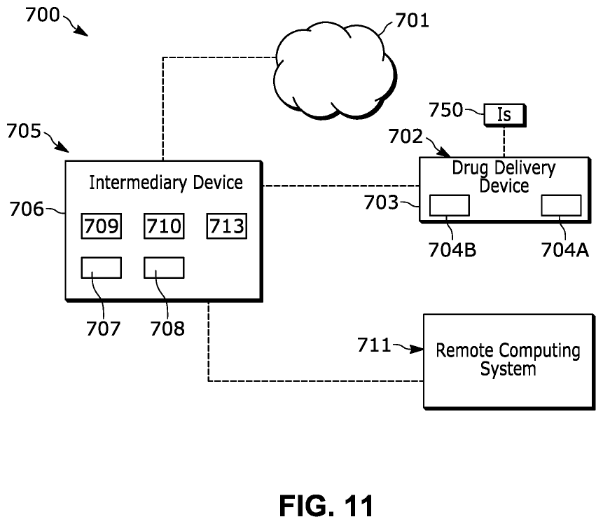

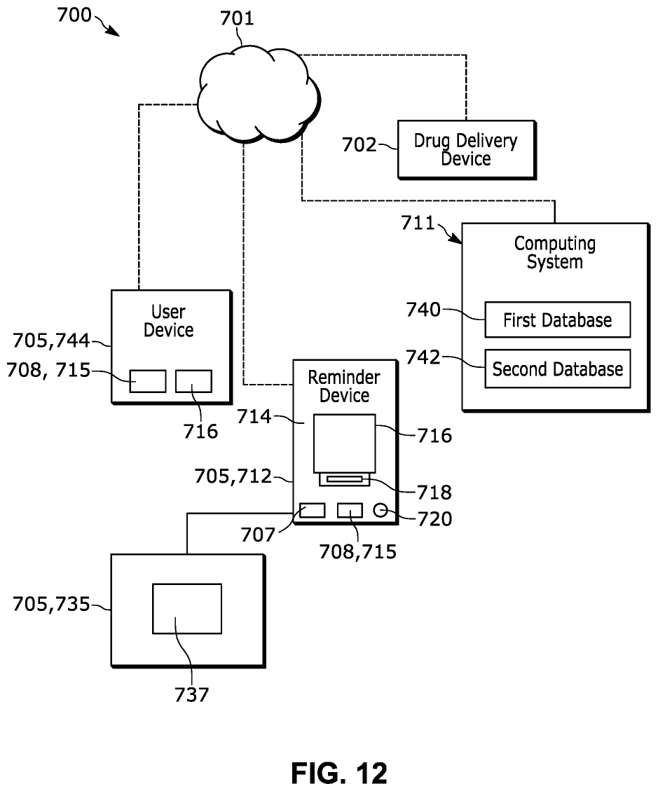

Referring now to FIGS. 11-13, a feedback system 700 for use with the dose of the drug product is depicted, and the feedback system 700 may include a reminder device 712, as depicted in FIG. 12. The reminder device 712 is very similar to the reminder device 12 of the reminder system 10 described above and depicted in FIGS. 1 and 2, with some additions. In particular, and unlike the reminder device 12 of FIGS. 1 and 2, the reminder device 712 of the feedback system 700 includes a microphone and a feedback mechanism, such as a speaker, to provide feedback to a user during the dose of the drug product (in addition to the reminder features), for example and as explained more below. As such, parts of the reminder device 712 of the feedback system 700 that are the same as parts of the reminder device 12 and the reminder system 10 of FIGS. 1 and 2 are numbered 700 more and are not explained again in detail for the sake of brevity. In addition, and unlike the reminder system 10 of FIGS. 1 and 2, the feedback system 700 further includes a drug delivery device to which the reminder device may be operatively coupled. So configured, feedback from one or more of the reminder device and/or a user device may be provided to the user about the dose of the drug product based on signals generated by the drug delivery device, for example, as also explained more below.

Referring now to FIG. 11, the feedback system 700 for use during a dose of a drug product includes a wireless network 701, a drug delivery device 702 including a body 703 and an audible indicator 704A for generating one or more audible signals. The audible indicator 704A may be a button or other retractable mechanism or any other part that creates a sound or “click” upon actuation of the drug delivery device 702, as is commonly understood by those having skill in the art. It will be further understood that the audible indicator 704A of the drug delivery device 702 may take the form of any other structure or part capable of generating one or more audible signals at the start of use, during use, or at the end of the use of the drug delivery device 702 and still fall within the scope of the present disclosure. The drug delivery device 702 may also include a visual indicator 704B for generating at least one visual signal. The visual indicator 704B may likewise take the form of any known structure or part of drug delivery devices capable of generating at least one visual signal, such as an light source or an LED light that turns “on” and/or emits light upon actuation of the drug delivery device 702, for example, and/or a change in state of the drug delivery device 702. In this example, the light source and/or LED light may change color and/or turn “off” during use and/or at the end of the use of the drug delivery device 702.

In addition, the feedback system 700 further includes an intermediary device 705 operatively coupled to the drug delivery device 702 and the wireless network 701. The intermediary device 705 includes a body 706 having a microphone 707 configured to amplify the one or more audible signals generated by the drug delivery device 702. Further, the intermediary device 705 also includes at least one feedback mechanism 708 carried by the body 706, a memory 709 carried by the body 706, and a processor 710 also carried by the body 706. The processor 710 is configured to execute logic stored on the memory 709 to cause the feedback mechanism 708 to provide feedback to the user about the dose of the drug product based on the one or more audible signals generated by the drug delivery device 702. In another example, the processor 710 is further configured to execute logic stored on the memory 709 to cause the feedback mechanism 708 to provide feedback to the user about the dose based on the at least one visual signal generated by the visual indicator 704B of the drug delivery device 702. In either case, the feedback may include one or both of an audio indication, such as a vocal indication, or a visual indication, as explained more below. In yet another example, the intermediary device 705 may further include a camera 713 that is also carried by the body 706. In this example, the processor 710 is further configured to cause the camera 713 to take a picture of the drug delivery device under certain conditions.

The one or more audible signals generated by the drug delivery device 702 include a start of dose “click” or sound when a button or other part of the drug delivery device 702 is actuated to initiate the dose of the drug product. The audible signal may also include an end of the dose (EOD) “click” or sound made by the button or another part of the drug delivery device 702 when the dose of the drug product is completed. In one example, intermediary device 705 is able to assess the character of the EOD “click” or sound to determine if the user lifts the drug delivery device 702 early from an injection site, for example. For instance, the EOD “click” or sound may be less muffled when not in contact with the user's skin at the injection site. Further, the audible signal generated by the drug delivery device 702 may also include a continuous clicking sound made during the dose of the drug product, for example. The intermediary device 705 is configured to discern and process a difference in sound between the start of the dose “click”, the EOD “click” and the continuous clicking.

As also explained more below, the intermediary device 705 may include the reminder device 712 (FIG. 12), a smart appliance, such as a smart refrigerator 735 to which the reminder device 712 may be attached (FIG. 12). The intermediary device 705 may also include any other smart user device, such as a smart phone (FIG. 12), a personal computer, a tablet, a smart speaker device, such as an Alexa smart speaker or similar product, and still fall within the scope of the present disclosure. In addition, the at least one feedback mechanism 708 of the intermediary device 705 may include a speaker and/or a display screen, as also explained more below.

Still referring to FIG. 11, the feedback system 700 also includes a remote computing system 711 having first and second databases (FIG. 12) and communicatively coupled to the intermediary device 705 via the wireless network 701. The processor 710 of the intermediary device 705 is further configured to collect data during the dose of the drug product and transmit the data to at least one of the databases of the remote computing system 711, as explained more below. In this way, data collected by the intermediary device 705 during the dose of the drug product of the drug delivery device 702 may be further analyzed to understand existing features, conditions, and/or user experiences of or related to the feedback system 700 and areas for improvement of the same, for example.

Referring now to FIG. 12, the feedback system 700 is again depicted with exemplary intermediary devices 705 included. In this example, the intermediary devices 705 include a reminder device 714, a smart appliance 735, and a user device 744, such as a smart phone. Each of these intermediary devices 705 at least includes the features described above relative to the intermediary device 705 of FIG. 11, for example, and other features as described herein.

More specifically, and like the reminder device 12 of FIGS. 1 and 2, the reminder device 712 includes a body 714 with the microphone 707, a feedback mechanism 708, such as a speaker 715, a display screen 716, which may include a touch screen, and a button 718. The display screen 716 may also be used as another type of feedback mechanism 708 of the reminder device 712, as explained more below. The reminder device 712 also includes a motion sensor 720 carried by the body 714. While the motion sensor 720 is disposed on a lower area of the body 714, the motion sensor 720 may alternatively be disposed on any part of the reminder device 712. Likewise, the microphone 707 and the feedback mechanism 708 may alternatively be disposed on any other area of the reminder device 712 and still fall within the scope of the present disclosure.

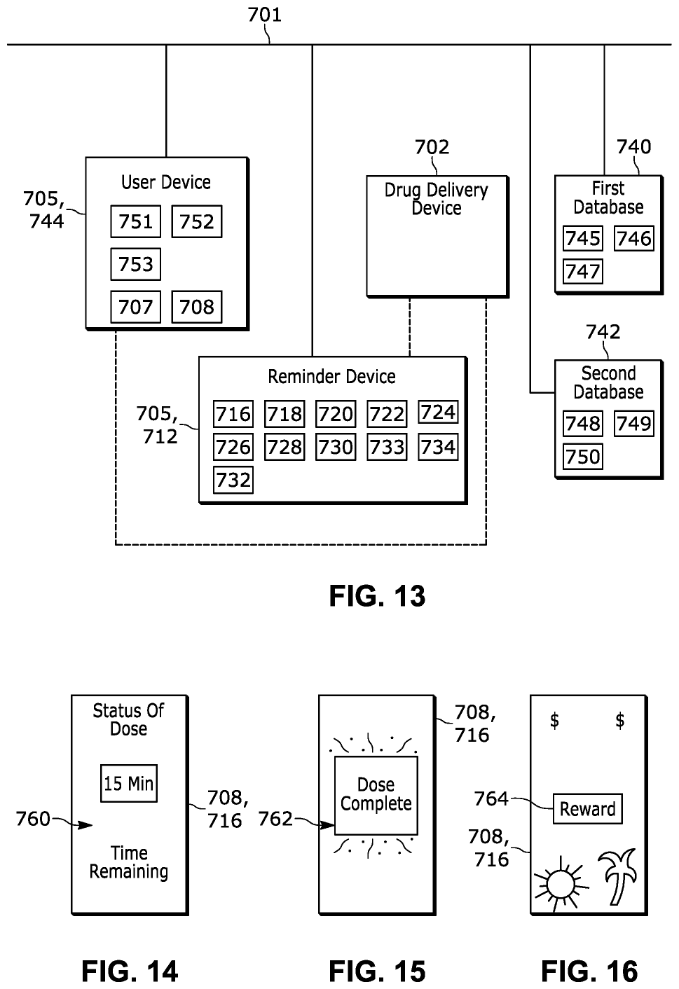

Referring now to FIG. 13, the reminder device 12 further includes an audible indicator 722 carried by the body 714 and for generating an audible alarm, a processor 724, and a memory 726, and each of the processor 724 and memory are also carried by the body 714. In addition, the memory 726 stores a dosing schedule and a reminder schedule, and each of the dosing schedule and the reminder schedule are based on user input provided via the touch screen 716 of the reminder device 712, as also described more above relative to the reminder device 12. Further, the dosing schedule includes at least one date for the dose inputted by the user, and the reminder schedule includes at least one time for activating the audible alarm of the audible indicator 722. The processor 724 is configured to access the dosing schedule and the reminder schedule and execute logic stored on the memory 26 to cause: (A) the touch screen 716 to display a countdown timer 727 for reminding the user of the at least one date for the dose. As explained more below, the countdown timer 727 updates as the at least one date for the dose approaches and comprises a visible indicator 728. The visible indicator 728 may include text or a color on a display of the touch screen 716, as explained more below. In addition, the visible indicator 728 may be activated upon actuation of the button 718 on the body 714 of the reminder device 712. Further, the visible indicator 728 may also be automatically activated at a set time before the at least one date of the dose and upon sensing motion via the motion sensor 720. Still further, the processor 724 may also execute logic stored on the memory 726 to cause the audible alarm of the audible indicator 722 to activate the at least one time for the dose, as also explained more below. As will be understood, the audible indicator 722 and the visible indicator 728 may be further feedback mechanisms of the reminder device 712.

In addition, the reminder device 712 includes a power source 730, which may include a battery or a wired connection, a power/charge port 732, a temperature timer 733, and a magnet or magnetic portion 734. The power/charging port 732 is used for powering the power source 730, such as a battery, of the reminder device 712, and no data can be accessed from the reminder device 712 via this port 732. Further, the magnet or magnetic portion 734 of the reminder device 712 may be attached to a surface of a refrigerated compartment 735, such as a smart refrigerator appliance, storing the drug product 737 (FIG. 12), such as the drug product for injection and of the drug delivery device 702. The temperature timer 733 may be activated for a period of time, such as 30 minutes, when the audible alarm 22 is activated, enabling the drug product 737 to be prepared for the dose, as also explained more below. In addition, the temperature timer 733 is configured to alert the user when the drug product has been out of the refrigerated compartment 735 for a sufficient time, allowing the drug product to reach a desired temperature, such as room temperature.

Referring back to FIG. 12, as noted the feedback system 700 also includes the wireless network 701, and the reminder device 712 may be operatively coupled to the wireless network 701 via a Wi-Fi router of the user. The reminder system 700 also includes at least one remote database operatively coupled to the wireless network 701. Specifically, the reminder device 712 supports an optional Wi-Fi connection that allows it to connect to an SMS server and the at least one remote database. In this example, the feedback system 700 includes the remote computing system 711 that includes a first remote database 740, such as a cloud mine database, for receiving data, such as a log of information about depersonalized user actions, collected by the processor 724 and stored in the memory 726 of the reminder device 712, in one example. In addition, the remote computing system 711 may also include a second remote database 742 for receiving data, such as a log of information about personalized user actions, again collected by the processor 724 and stored in the memory 726 of the reminder device 712 in this example.

As further depicted in FIG. 13, the first remote database 740 includes a processor 745, a memory 746, and a network interface 747, and the second remote database 742 also includes a processor 748, a memory 749, and a network interface 750. So configured, the processors 745, 748 of each of the first and second remote databases 740, 742 receive data, such as the logs of information, from one or more of the reminder device 712 or the user device 744 (also in FIG. 12), for example, and may generate reports based on the collected information. As also depicted in FIG. 13, and as will be understood, the user device 744 also includes at least a processor 751, a memory 752, and a network interface 753 operatively coupled to the wireless network 701. The user device 744 also further includes the microphone 707 and the at least one feedback mechanism 708, such as the speaker 715 or the display screen 716, as depicted in FIG. 12.

In view of the foregoing it will be understood that the feedback system 700 may provide many types of feedback to the user using various types of feedback mechanisms of various intermediary devices based on one or more signals generated by the drug delivery device 702 during the dose of the drug product. For example, and in one example, the at least one feedback mechanism 708 is the speaker 715 of any one of the intermediary device 705, the reminder device 712, and the user device 744. The processor 710, 724, 751 of the intermediary device 705, the reminder device 712, and the user device 744, respectively, is configured to cause the speaker 708 to provide feedback, such as an audio indication, including one or more of: (1) a rebroadcast of the audible signal generated by the drug delivery device 702 at a sound level higher than the sound level of the audible signal initially generated by the drug delivery device 702; (2) a vocal instruction indicating one or more of the status of the dose of the drug product or an estimate of a remaining time for the dose of the drug product, (3) an indication that the drug delivery device 702 is lifted from the injection site 750 (FIG. 11) based on an acoustic character of the audible signal generated from the drug delivery device 702; and (4) a vocal instruction that the dose of the drug product by the drug delivery device 702 is complete based on a number of audible signals generated by the drug delivery device 702.

When the feedback includes the rebroadcast of the audible signal generated by the drug delivery device 702 at a sound level higher than the sound level of the initial audible signal, the rebroadcast and/or sound may be tailored to the environment of the user, for example. For instance, if the intermediary device 705 detects a background sound power or level that is at about 50 dB, the intermediary device 705 may choose to provide the feedback to the user, e.g., provide the rebroadcast of the audible signal, at level of about 53 dB so as to only be as loud as necessary without startling the user of the drug delivery device 702 during the dose of the drug product. Said another way, if the background level of sound is about 50 dB, the intermediary device 502 may provide feedback to the user via the audio indication that is at a level of sound greater than the background level of sound, such as about 53 dB in this example. In addition, when the feedback includes the vocal instruction, the intermediary device 705 may provide the vocal instruction in a language of the user's choosing, regardless of a region.

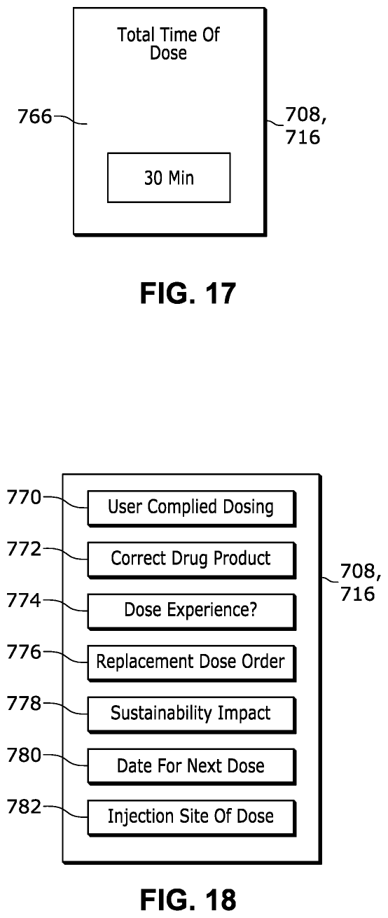

Referring now to FIGS. 14-17, in another example the at least one feedback mechanism 708 is the display screen 708, 716 of the intermediary device 705, the reminder device 712, and the user device 744. In this example, the processor 710, 724, 751 of each of the intermediary device 705, the reminder device 712, and the user device 744 is configured to cause the display screen 708, 716 to provide feedback including a visual indication on the display screen 708, 716. The visual indication includes one or more of a countdown bar 760 for providing the status of dose, as depicted in FIG. 14, and a celebration indication 762 when the dose of the drug product is completed, as depicted in FIG. 15. In addition, the visual indication may additionally or alternatively include one or more of a distracting reward indication 764 to minimize user perception of pain, as depicted in FIG. 16, and a report 766 providing a total time for the dose of the drug product to be completed, as depicted in FIG. 17.

In another example, the at least one feedback mechanism 708, 722 is the audible indicator, such as the speaker 715, and the feedback provided based on the one or more audible signals generated from the drug delivery device 702 includes an audio indication, such as a vocal indication. The vocal indication may include one or more of: (1) a total time for the dose of the drug; (2) a celebration of completion of the dose of the drug product; (3) a countdown of time remaining in the dose of the drug product; (4) an indication that the drug delivery device 702 may be tampered with or counterfeit; and/or (5) a confirmation that the drug delivery device is locked out after completion of the dose of the drug product.

When the vocal indication includes that the drug delivery device 702 may be tampered with or counterfeit, the intermediary device 705 is able to identify such tampered or counterfeit drug delivery devices 702 based on comparing the one or more audible signals from the drug delivery device 702 to a designed audible signal of the actual, correct drug delivery device 702. If the sound of the actual audible signal received from the drug delivery device 702 matches the sound of the designed audible signal of the drug delivery device 702, no potential tampered with or counterfeit drug delivery device indications is provided by the intermediary device 705. However, if the sound of the actual audible signal received from the drug delivery device 702 is different from the sound of the designed audible signal of the drug delivery device 702, the intermediary device 705 provides the feedback to the user in the form of the vocal indication in this example.

In another example, the processor 710, 724, 751 of each of the intermediary device 705, the reminder device 712, and the user device 744 of the feedback system 700 is further configured to execute logic stored on the memory 709, 726, 752 to cause the processor 710, 724, 751 to collect data during the dose of the drug product and while receiving the one or more audible signals from the drug delivery device 702. The data collected may include one or more of: (1) data regarding statistics on training efficacy of administering the dose of the drug product; (2) data regarding total time for completion of the dose of the drug product; (3) data regarding an environment in which the dose of the drug product is administered; and (4) data regarding disposal of the drug delivery device 702. In addition, the processor 710, 724, 751 is further configured to execute logic stored on the memory 709, 726, 752 to cause the processor 710, 724, 751 to transmit collected data to the at least one remote database 740, 742 of the remote computing system 711.

The data collected and transmitted may be used for a variety of purposes. For example, the data collected regarding the total time for completion of the dose of the drug product may be used to improve an understanding of product consistency. In addition, data collected on the environment of the dose, such as the injection, may include sound, light and location of the environment of the user. This data may then be used to improve the user environment design and allow for a more robust human factors definition of user needs for future drug products, accessories, and lifecycle management efforts. Still further, data regarding disposal of the drug delivery device 702 may be used to better optimize container distribution and collection, such as when the user may need a replacement and a collection of a full container, for example.

In yet another example, and referring now to FIG. 18, the at least one feedback mechanism 708 is the display screen 716, and the feedback provided by the feedback mechanism 708 includes one or more of: (1) an indication 770 a user complied with a prescribed dosing interval; (2) an indication 772 confirming a correct drug product was administered, e.g., such as based on sound and time intervals of the one or more audible signals; (3) a solicitation 774 regarding dose experience after the dose is completed; (4) an indication 776 of a reorder of a replacement dose of the drug product upon sensing the drug delivery device 702 stalled or a user lifted the drug delivery device 702 from an injection site prematurely; (5) an estimate of sustainability impact 778 based on a user's geolocation at a time of the dose compared to an alternative injection site; (6) an indication 780 providing a date for a next dose of the drug product; and (7) a solicitation 782 regarding what injection site was chosen after an end of dose click to provide guidance at the time of the next injection regarding a different injection site. In another example, and as will be understood, the at least one feedback mechanism may additionally and/or alternatively include an audible indicator, such as the speaker 715, and each of the indications and solicitations displayed in FIG. 18 and described above may additionally and/or alternatively be provided via the audio indication, such as the vocal instruction or indication.

In one example, when the feedback includes the estimate of sustainability impact based on the user's geolocation, the estimate may be based on comparing the distance of the user's residence location in which dose of the drug product is administered to a point of care location the user would need to travel to receive the dose of the drug product if unable to administer at the user's residence location. More specifically, if the user lives 50 miles from the point of care location, administering the dose of the drug product via the drug delivery device 702 at the user's residence location saves the user from needing to take that trip to the point of care location. As a result, the user avoids associated emissions from the trip and travel.

In another example, when the feedback includes the solicitation regarding what injection site was chosen, the data received from the solicitation may be used to provide guidance to the user at the time of the next dose of the drug product, e.g., the injection, regarding what injection site will be appropriate. For example, users should typically select a different time for each dose of the drug product, such as for each injection. The user may make a note about how the injection at a particular injection site for a particular dose of the drug product went, such as was the dose of the drug product at that site abnormally painful, hard to pinch enough skin, and/or awkward to hold the drug delivery device 702. This data then allows the intermediary device 705 to provide potentially further feedback including correction suggestions to a better injection site for the user and/or what accessories the user should use when administering the dose of the drug product.

In view of the foregoing, it will be appreciated that that the feedback system 700 and foregoing features of the same may operate according to the following method. Specifically, a method of providing feedback to the user during a dose of a drug product, may include receiving one or more audible signals generated by the drug delivery device 702 via the intermediary device 705 and providing feedback to the user about a dose of a drug product of the drug delivery device 702 via the feedback mechanism 708 of the intermediary device 705 based on the one or more audible signals, the feedback including one or more of an audio indication, such as the vocal indication, or a visual indication. The method may also include collecting data, via the processor 710 of the intermediary device 705, while receiving the one or more audible signals from the drug delivery device 702, the data including one or more of: (1) data regarding statistics on training efficacy of administering the dose of the drug product; (2) data regarding total time for completion of the dose of the drug product; (3) data regarding an environment in which the dose of the drug product is administered; and (4) data regarding disposal of the drug delivery device 702. The method may also include transmitting the data, via the processor 710 of the intermediary device 705, to the at least one remote database 740, 742 via the wireless network 701.

In some examples, receiving one or more audible signals generated by the drug delivery device 702 via the intermediary device 705 may include receiving one or more audible signals generated by the drug delivery device 702 via the microphone 707 of the intermediary device 705. In another example, providing feedback to the user about the dose of the drug product of the drug delivery device 702 via the feedback mechanism 708 of the intermediary device 705 based on the one or more audible signals may include providing feedback to the user about the dose of the drug product of the drug delivery device 702 via the speaker 715. In this example, the feedback may include the audio indication, and the audio indication may include a vocal instruction including one or more of: (1) a rebroadcast of the audible signal generated by the drug delivery device 702 a sound level higher than the sound level of the audible signal initially generated by the drug delivery device 702 via the speaker 715; (2) a vocal instruction indicating one or more of the status of the dose of the drug product or an estimate of the remaining time for the dose of the drug product in the drug delivery device 702; (3) an indication that the drug delivery device 702 is lifted from an injection site based on an acoustic character of the audible signal generated from the drug delivery device 702; and (4) a vocal instruction that the dose of the drug product by the drug delivery device 702 is complete based on a number of audible signals generated by the drug delivery device 702.

In another example, providing feedback to the user about the dose of the drug product of the drug delivery device 702 via the feedback mechanism 708 of the intermediary device 705 based on the one or more audible signals may include providing feedback to the user about a dose of a drug product of the drug delivery device 702 via a display screen 716, and the feedback includes a visual indication on the display screen 716. In this example, the visual indication includes one or more of a countdown bar for providing the status of the dose, a celebration indication when the dose of the drug product is completed, a distracting reward indication to minimize user perception of pain, and a report providing a total time for the dose of the drug product to be completed.

In view of the foregoing, it will be understood that the feedback system 700 and related methods have several advantages. For example, the intermediary device 705 of the feedback system 700 and method is able to transform existing audible spectra from existing feedback mechanisms on known drug delivery devices into electromagnetic spectra via the wireless connection of the intermediary device without having to add a wireless module to the drug delivery device. In this way, helpful feedback via the intermediary device 705, such as the reminder device 712 or the smart user device 744, is provided to the user during the dose of the drug product in a cost effective and sustainable manner. Further, the usability of the drug delivery devices is enhanced at several stages, providing both feedback and training to the user. For example, initial training using the drug delivery device 702 and ongoing use of the drug delivery device 702 is enhanced with the ongoing feedback of the feedback system 700 and methods of the present disclosure. In addition, the feedback system 700 and method are able to assist with continuous improvement of the use of the drug delivery device 702 while remaining cost effective and environmentally sustainable. Still further, voluntary data collection may also provide the ability for payers to receive assurances that preventative therapies are being administered in a manner that lowers payer fiscal risk and allows for improved value proposition for therapy and drug delivery entities.

The above description describes various devices, systems, and methods related to reminders and feedback for doses of a drug product. The device, components, subsystems, or methods can be used with a drug product, for example certain methods according to the present disclosure may include injection of a drug product after one or more reminders, where the drug products can include but are not limited to the drug products identified below as well as their generic and biosimilar counterparts. The term drug product, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug product may be in liquid form, a lyophilized form, or in a reconstituted from lyophilized form. The following example list of drug products should not be considered as all-inclusive or limiting.

The drug product will be contained in a reservoir of a drug delivery device. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL 1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-aαß7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIia receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL 15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Rα mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL 12 mAb (ABT-874); anti-IL 12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGB mAb (MDX-1307); anti-mesothelin dsFv-PE38 conjugate (CAT-5001); anti-PD1mAb (MDX-1106 (ONO-4538)); anti-PDGFRa antibody (IMC-3G3); anti-TGFß mAb (GC-1008); anti-TRAIL Receptor-2 human mAb (HGS-ETR2); anti-TWEAK mAb; anti-VEGFR/FIt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRASG12C small molecule inhibitor, or another product containing a KRASG120 small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA)×anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1× IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33× anti-CD3 BITE® (bispecific T cell engager) construct. In some embodiments, the drug delivery device may contain or be used with AMG 404 or another product containing a human anti-programmed cell death-1(PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3)×anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP×4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19× CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3× epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33× anti-CD3 BiTE® (bispecific T cell engager) construct. In some embodiments, the drug delivery device may contain or be used with AMG 701 or another product containing a half-life extended (HLE) anti-B-cell maturation antigen (BCMA)× anti-CD3 BiTE® (bispecific T cell engager) construct. In some embodiments, the drug delivery device may contain or be used with AMG 757 or another product containing a half-life extended (HLE) anti-delta-like ligand 3 (DLL3)× anti-CD3 BiTE® (bispecific T cell engager) construct. In some embodiments, the drug delivery device may contain or be used with AMG 910 or another product containing a half-life extended (HLE) epithelial cell tight junction protein claudin 18.2× CD3 BiTE® (bispecific T cell engager) construct.

Although the device, system, and method have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A reminder device for reminding a user when to dose a drug product, the reminder device comprising:
a body having a touch screen and a button;
a motion sensor carried by the body;
an audible indicator carried by the body for generating an audible alarm;
a memory carried by the body and storing a dosing schedule and a reminder schedule, each of the dosing schedule and the reminder schedule based on user input via the touch screen, the dosing schedule including at least one date for a dose of the drug product, and the reminder schedule including at least one time for activating the audible alarm; and
a processor carried by the body and configured to access the dosing schedule and the reminder schedule and execute logic stored on the memory to cause
(A) the touch screen to display a countdown timer for reminding the user of the at least one date for the dose, the countdown timer updating as the at least one date for the dose approaches and comprising a visible indicator that is activated: (1) upon actuation of the button on the body; or (2) automatically at a set time before the at least one date of the dose and upon sensing motion via the motion sensor, and
(B) the audible alarm to activate at the at least one time for the dose.

2. The reminder device of claim 1, wherein, after the dose is due, the logic is further executable by the processor to automatically change the touch screen to a color indicating the dose is past due.

3. The reminder device of claim 1, wherein the body includes one or more of: (1) a magnet adapted to attach to a surface of a refrigerated compartment storing the drug product; or (2) a surface adapted to be coupled to a stand disposed near the drug product.

4. The reminder device of claim 1, wherein each of the dosing schedule and the reminder schedule may include a frequency, such as weekly, bi-monthly, monthly, twice weekly, once every number of months, or once every number of days, the number able to be set to a desired value.

5. The reminder device of claim 1, further comprising at least one of (a) through (c) as follows:
(a) a user-configured wireless communication module for wireless transmission of a text message reminder to a user device, the text message transmitted one or more of: (1) at the at least one date the dose is due, or (2) when the dose is past due;
(b) a log of information about depersonalized user actions collected by the processor and stored in the memory, and a user-configured wireless communication module for wireless transfer of the log of information to a remote database;
(c) a temperature timer configured to alert the user when the drug product has been out of a refrigerated compartment for a sufficient time to allow the drug product to reach a desired temperature, such as room temperature.

6-7. (canceled)

8. The reminder device of claim 1, wherein, after the dose of the drug product is indicated to be taken, the logic is further executable by the processor to provide an option to reorder on the touch screen, and the option to reorder is activated based on user input via the touch screen.

9-15. (canceled)

16. A method of managing reminders for a user to dose a drug product, the method comprising:
upon actuating a reminder device, displaying a countdown timer on a touch screen of the reminder device, the countdown timer for reminding a user of at least one date for a dose of a drug product set by the user;
updating the countdown timer as the at least one date for the dose approaches;
activating a visible indicator of the countdown timer: (1) upon actuating a button of the reminder device; and (2) automatically at a set time before the at least one date for the dose and upon sensing motion via a motion sensor; and
activating an audible alarm of an audible indicator of the reminder device at a time set by the user.

17. The method of claim 16, further comprising storing, via a memory of the reminder device, one or more of a dosing schedule and a reminder schedule based on input from the user, the dosing schedule including the at least one date for a dose of the drug product, and the reminder schedule including the time for activating the audible alarm.

18. The method of claim 16, further comprising automatically changing the touch screen of the reminder device to a color indicating the dose is past due, providing another reminder to the user.

19. The method of claim 16, further comprising at least one of (a) through (c), as follows:
(a) transmitting, via a processor of the reminder device, a text message reminder to a user device via a wireless network at the at least one date for the dose or when the dose is past due, the text message reminder including the at least one date for the dose;
(b) actuating the touch screen on the reminder device in response to the audible alarm to initiate a snooze function for the audible alarm and for a period of time, and automatically reactivating the audible alarm after the period of time expires;
(c) activating a temperature timer configured to alert the user when the drug product has been out of a refrigerated compartment for a sufficient time, allowing the drug product to reach a desired temperature, such as room temperature.

20-21. (canceled)

22. A feedback system for use with a dose of a drug product, the feedback system comprising:
a wireless network;
a drug delivery device including a body and an audible indicator carried by the body and for generating one or more audible signals;
an intermediary device operatively coupled to the drug delivery device and the wireless network, the intermediary device including:
a body having a microphone configured to amplify the one or more audible signals generated by the drug delivery device;
at least one feedback mechanism carried by the body;
a memory carried by the body; and
a processor carried by the body and configured to execute logic stored on the memory to cause the at least one feedback mechanism to provide feedback to the user about a dose of the drug product based on the one or more audible signals generated by the drug delivery device, the feedback including one or more of an audio indication or a visual indication.

23. The system of claim 22, wherein the intermediary device is one of a reminder device, a smart phone, a personal computer, a tablet, a smart appliance, or a smart speaker device, and the at least one feedback mechanism includes one or more of a speaker or a display screen.

24. The system of claim 22, wherein the intermediary device is a reminder device, the reminder device further comprising a touch screen disposed on the body, a motion sensor carried by the body, and an audible indicator carried by the body for generating an audible alarm, the memory storing a dosing schedule and a reminder schedule, the dosing schedule including at least one date for a dose of the drug product, and the reminder schedule including at least one time for activating the audible alarm, the processor further configured to access the dosing schedule and the reminder schedule and further execute logic stored on the memory to cause: (A) the touch screen to display a countdown timer for reminding the user of the at least one date for the dose, the countdown timer updating as the at least one date for the dose approaches and comprising a visible indicator that is activated: (1) upon actuation of a button on the body; or (2) automatically at a set time before the at least one date of the dose and upon sensing motion via the motion sensor, and (B) the audible alarm to activate at the at least one time for the dose.

25. The system of claim 22, wherein the feedback is an audio indication and includes one or more of: (1) a rebroadcast of the one or more audible signals generated by the drug delivery device at a sound level higher than the sound level of the one or more audible signals initially generated by the drug delivery device; (2) a vocal instruction indicating one or more of the status of the dose of the drug product or an estimate of a remaining time for the dose of the drug product, (3) an indication that the drug delivery device is lifted from an injection site based on an acoustic character of the audible signal generated from the drug delivery device; and (4) a vocal instruction that the dose of the drug product by the drug delivery device is complete based on a number of audible signals generated by the drug delivery device.

26. The system of claim 22, wherein the at least one feedback mechanism is at least one of (a) to (b), as follows:
(a) a display screen, and the feedback is a visual indication on the display screen, the visual indication including one or more of a countdown bar for providing the status of the dose, a celebration indication when the dose of the drug product is completed, a distracting reward indication to minimize user perception of pain, and a report providing a total time for the dose of the drug product to be completed;
(b) one of a speaker or a display screen, and the feedback provided by the feedback mechanism includes one or more of: (1) an indication a user complied with a prescribed dosing interval; (2) an indication confirming a correct drug product was administered based on sound and time intervals of the one or more audible signals; (3) a solicitation regarding dose experience after the dose is completed; (4) an indication of a reorder of a replacement dose of the drug product upon sensing the drug delivery device stalled or a user lifted the drug delivery device from an injection site prematurely: (5) an estimate of sustainability impact based on a user's geolocation at a time of the dose compared to an alternative injection site: (6) an indication providing a date for a next dose of the drug product; and (7) a solicitation regarding what injection site was chosen after an end of dose click to provide guidance at the time of the next injection regarding a different injection site.

27. The system of claim 22, wherein the feedback is an audio indication including a vocal indication, the vocal indication including one or more of: (1) a total time for the dose of the drug; (2) a celebration of completion of the dose of the drug product; (3) a countdown of time remaining in the dose of the drug product; (4) an indication that the drug delivery device may be tampered with or counterfeit; and (5) a confirmation that the drug delivery device is locked out after completion of the dose of the drug product.

28. The system of claim 22, wherein the drug delivery device further comprises a visual indicator carried by the body and for generating at least one visual signal, and the processor is further configured to execute logic stored on the memory to cause the feedback mechanism to provide feedback to the user about the dose of the drug product based on the at least one visual signal generated by the drug delivery device, the feedback including one or more of a vocal indication or a visual indication.

29. The system of claim 22, further comprising at least one remote database operatively coupled to the wireless network, wherein the processor is further configured to execute logic stored on the memory to cause the processor to collect data during the dose of the drug product and while receiving the one or more audible signals from the drug delivery device, the data including one or more of: (1) data regarding statistics on training efficacy of administering the dose of the drug product; (2) data regarding total time for completion of the dose of the drug product; (3) data regarding an environment in which the dose of the drug product is administered; and (4) data regarding disposal of the medication device, and wherein the processor is further configured to execute logic stored on the memory to cause the processor to transmit collected data to the at least one remote database.

30. The system of claim 22, wherein the one or more audible signals generated by the drug delivery device includes one or more of a start of dose click, an end of dose (EOD) click, and a continuous clicking during the dose, and the intermediary device is configured to discern and process a difference in sound between the start of the dose click, the EOD click, and the continuous clicking.

31. The system of claim 22, wherein the intermediary device further comprises a camera carried by the body, and the processor is further configured to execute logic stored on the memory to cause the camera to take a picture of the drug delivery device in response to the one or more audible signals indicating one of a stalled dose of drug product or an absence of an audible signal indicating an end of dose of the drug product.

32-37. (canceled)
